(19) United States
(12) Patent Application Publication
(10) Pub. No.: US 2026/0335263 A1
(43) Pub. Date: Oct. 29, 2026

(54) USE OF TROPIC ACID AND DERIVATIVE THEREOF IN PREPARATION OF DRUG FOR TREATING PSORIASIS

(71) Applicant: GUANGDONG PHARMACEUTICAL UNIVERSITY, Guangzhou, Guangdong (CN)

(72) Inventors: Changfu WANG, Guangzhou, Guangdong (CN); Ruiqiang XIE, Harbin, Heilongjiang (CN); Ping XIN, Daqing, Heilongjiang (CN)

(73) Assignee: GUANGDONG PHARMACEUTICAL UNIVERSITY, Guangzhou, Guangdong (CN)

(21) Appl. No.: 19/122,455

(22) PCT Filed: Oct. 20, 2023
(86) PCT No.: PCT/CN2023/125517

§ 371 Date: Apr. 18, 2025

(30) Foreign Application Priority Data
Oct. 25, 2022 (CN) 202211309894.3

(51) Int. Cl. A61K 31/192; A61K 9/00; A61K 9/06; A61K 9/20; A61K 47/02; A61K 47/06; A61K 47/10; A61K 47/12; A61K 47/26; A61P 17/06; C07C 65/01

(52) U.S. Cl. CPC A61K 31/192; A61K 9/0014; A61K 9/0019; A61K 9/06; A61K 9/2009; A61K 9/2013; A61K 9/2018; A61K 9/2027; A61K 9/2054; A61K 47/02; A61K 47/06; A61K 47/10; A61K 47/12; A61K 47/26; A61P 17/06; C07C 65/01

(57) ABSTRACT

The use of tropic acid and a derivative thereof in the preparation of a drug for treating psoriasis. It is found that tropic acid and the derivative thereof can significantly improve skin damage of a psoriasis animal model. The tropic acid and the derivative thereof have low toxicity, fast action, a short treatment course, a small dosage, a low recurrence rate and is convenient to use, and the dosage forms for external use, oral administration, injection, etc. are taken into consideration, such that it is possible to adapt to patients with different degrees and different types of psoriasis. It is also found that compounds III and IV obtained by means of structural modification have a significantly better therapeutic effect than tropic acid. The small molecule drugs used are easily obtained, have a low price and stable properties, are convenient to store and transport, and have broad application prospects.

### CROSS-REFERENCE TO RELATED APPLICATION

This application claims the priority benefit of China patent application No. 2022113098943, filed on Oct. 25, 2022, which is incorporated herein by reference in its entirety.

### TECHNICAL FIELD

The present disclosure relates to the field of pharmaceutical technology, and particularly relates to the use of tropic acid and its derivatives in the manufacture of a medicament for treating psoriasis and a medicament for treating psoriasis.

### BACKGROUND ART

Psoriasis is a complex, multifactorial, chronic and inflammatory autoimmune skin disease, characterized clinically by well-defined erythema and scales. The pathological manifestations in the skin of psoriasis patients include excessive proliferation of keratinocytes, infiltration of inflammatory cells, and neovascularization. Clinical manifestations include excessive epidermal proliferation, incomplete keratinization, and chronic inflammatory response in the dermis. Psoriasis has a high recurrence rate, and patients often require long-term or repeated medication. When the disease occurs, it may be accompanied by systemic manifestations, with the elbow, knee, hand, sacrococcygeal region, and scalp being the main sites of pathological plaques. According to the clinical characteristics, psoriasis can be classified into types of vulgaris, arthropathic, pustular, and erythrodermic, and the vulgaris type being the most common. The pathogenesis of psoriasis is very complex and has not been fully elucidated. Currently, it is believed that factors such as race, genetics, and environment are closely related to the occurrence and progression of this disease. According to epidemiological survey data on psoriasis, the global incidence rate of psoriasis is about 2%, and the prevalence of psoriasis in different countries and regions ranges from 0.09% to 11.4%. Psoriasis can occur at any age, with an adult prevalence ranging from 0.51% to 11.43% and a child prevalence ranging from 0% to 1.37%. There are currently about over 6 million psoriasis patients in China. Psoriasis is a systemic disease, non-infectious, and mainly affecting the skin and joints, with 20% to 30% of patients experiencing joint damage. Moderate and severe psoriasis patients have an increased risk of metabolic syndrome and atherosclerotic cardiovascular disease. In addition, social stigma and personal psychological issues seriously affect the quality of life of patients.

At present, there are many drugs for treating psoriasis, mainly including monoclonal antibodies, steroids, immunosuppressants, retinoids, JAK/STAT signaling pathway inhibitors, nonsteroidal anti-inflammatory drugs, anti-tumor drugs, phosphodiesterase inhibitors, other topical drugs, physical therapy, and traditional Chinese medicine. Monoclonal antibody drugs include IL-23 inhibitors, IL-17A targeted drugs, anti-CD monoclonal antibody drugs, anti-tumor necrosis factor drugs, IL-6 inhibitors, and IL-12/IL-23 inhibitors. Among them, IL-23 inhibitors include guselkumab, tildrakizumab, and risankizumab. Monoclonal antibodies targeting IL-17A include secukinumab, ixekizumab, brodalumab, and netakimab. The anti-CD monoclonal antibody drugs mainly include efalizumab, alefacept, and rituximab. Anti tumor necrosis factor drugs include etanercept, adalimumab, infliximab, certolizumab pegol, golimumab, and onercept. IL-6 inhibitors such as tocilizumab and IL-12/IL-23 inhibitor ustekinumab. These drugs are mainly used for the systemic treatment of moderate to severe psoriasis, some of which have just been launched and are in the clinical trial stages, requiring more evidence-based support. This type of drug also has many adverse reactions and is not suitable for long-term or repeated use by psoriasis patients, such as increasing the risk of infection, worsening Crohn's disease, and the risk of emerging or worsening diseases of tumors, lupus, malignant tumors, infections, and heart failure. Other adverse reactions include respiratory inflammation, fever, rash, joint inflammation, and lymph node enlargement. Some drugs such as efalizumab and alefacept have been withdrawn from the market due to serious adverse effects. In addition, due to the high development cost and high price of these drugs, it is difficult for patients to afford them. The steroid hormone betamethasone and immunosuppressive vitamin D3 derivatives calcipotriol, tacrolimus, abatacept, and cyclosporine are effective in treating psoriasis, but they have serious hormone dependence and immune system side effects. Retinoids are particularly suitable for pustular and erythrodermic psoriasis, and the main adverse reactions include effects on mucocutaneous, reproductive system, bones and muscles, liver function, blood lipids, and skin irritation. Tazarotene cream is a first-line drug to treat localized plaque psoriasis, but due to its strong stimulation, it is not suitable for use on the face, around the eyes, external genitalia, and skin folds. Targeted topical JAK/STAT signaling pathway inhibitor tofacitinib can be used for the treatment of arthropathic psoriasis, with adverse reactions such as increased infection rate of herpes zoster or inducing thromboembolism. JET-052 ointment may be effective in treating inflammatory lesions of psoriasis. Non steroidal anti-inflammatory drug benvitimod is independently developed in China and has achieved good therapeutic effects in both animal experiments and clinical trials. However, there are also some adverse reactions that become significant with increasing drug concentration. Overall, the adverse reactions are relatively small and mostly mild skin irritation, such as itching, contact dermatitis, folliculitis, etc. The anti-tumor drug methotrexate is commonly used for erythrodermic psoriasis, arthropathic psoriasis, and extensive chronic plaque psoriasis. During the treatment process, it can cause serious adverse reactions such as liver toxicity, marrow suppression, and pulmonary fibrosis. The phosphodiesterase inhibitor apremilast showed good efficacy and tolerability in treating arthropathic psoriasis, with common side effects such as diarrhea, nausea, and headache. Other topical treatments include moisturizers, keratoplastics, keratolytics, anthralin, and tar preparations. Physical therapy includes narrowband ultraviolet radiation, A-band ultraviolet radiation, 308 nm excimer laser, hot spring baths, and seawater baths. Traditional Chinese medicine therapy includes internal and external treatments, mainly including oral Chinese medicine decoctions or formulas, external Chinese medicine fumigation and acupuncture. Chinese medicine therapy has significant efficacy and few adverse reactions. However, many doctors have different opinions on the etiology and pathogenesis of psoriasis, and have not yet formed a unified treatment plan. It has a long course of treatment and is easy to recur. Some treatments are often unacceptable for patients because of inconvenient use and sensory discomfort such as drug odor or color.

In summary, a large population suffering from psoriasis, and although there are many types of therapeutic drugs or methods, their efficacy is limited. Psoriasis patients often require long-term, repeated, or even lifelong treatment. Although some drugs may achieve temporary efficacy, the short-term recurrence rate is high. Most drugs are often constrained by their inherent toxicity and selectivity, inevitably leading to various systemic and local adverse reactions. In addition, most of the existing therapeutic agents are topical medications, which are inconvenient to use, and there are few oral medications. In response to the above issues, there is an urgent need to develop therapeutic drugs with features of rapid onset, short course, small dosage, minimal adverse reactions, low recurrence rate, low price, and convenient to use. Meanwhile, the development of external, oral, and injectable drug formulations should be considered to adapt to patients with different degrees and types of psoriasis. The efficacy of DL-tropic acid (also known as 2-phenyl-3-hydroxypropionic acid) and its derivatives in treating psoriasis has been screened and confirmed through extensive animal experiments in the early stage of the present invention. Tropic acid is an intermediate for the synthesis of atropine, with low raw material prices. Currently, there are no reports on the prevention and treatment of psoriasis using tropic acid and its derivatives.

### DISCLOSURE OF THE INVENTION

In one aspect, the present disclosure provides the use of tropic acid and a derivative thereof, or a pharmaceutically acceptable salt, a solvate, an enantiomer, a diastereoisomer, or a tautomer thereof, or a mixture thereof in any proportion in the manufacture of a medicament for the prevention and/or treatment of psoriasis, wherein the tropic acid and a derivative thereof have a structure represented by the following formula A:

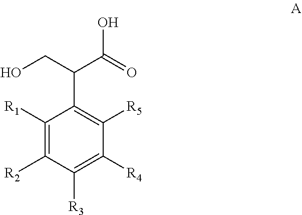

wherein, R₁-R₅ are each independently selected from —H and —OH or C₁-C₆ alkoxy.

In one embodiment, R₁-R₅ are each independently selected from —H or —OH.

In one embodiment, the tropic acid and a derivative thereof are selected from the following compounds represented by formulas I to IV:

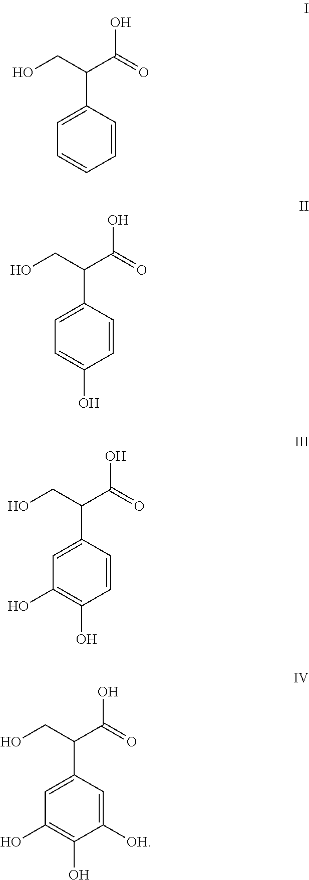

wherein, formula I is DL-tropic acid, formula II is 4-hydroxy-α-(hydroxymethyl)benzeneacetic acid, formula III is 3,4-dihydroxy-α-(hydroxymethyl)benzeneacetic acid, and formula IV is 3,4,5-trihydroxy-α-(hydroxymethyl)benzeneacetic acid.

The tropic acid and its derivatives described in the present disclosure have a significant improvement effect on the skin tissue morphology and histopathology of a psoriasis mouse model induced by imiquimod cream.

In one embodiment, the psoriasis is selected from one or more of the group consisting of vulgaris psoriasis, arthropathic psoriasis, erythrodermic psoriasis, and pustular psoriasis.

In the second aspect, the present disclosure provides a medicament for preventing and/or treating psoriasis, comprising tropic acid and a derivative thereof, or a pharmaceutically acceptable salt, a solvate, an enantiomer, a diastereoisomer, or a tautomer thereof, or a mixture thereof in any proportion.

The medicament for preventing and/or treating psoriasis provided by the present disclosure has significant effects.

In one embodiment, the tropic acid and a derivative thereof, or a pharmaceutically acceptable salt, a solvate, an enantiomer, a diastereoisomer, or a tautomer thereof, or a mixture thereof in any proportion are used as active ingredient in the medicament of the present disclosure; preferably, used as main active ingredient; more preferably, used as the sole active ingredient.

For the above-mentioned use and medicament, tropic acid and a derivative thereof, or a pharmaceutically acceptable salt, a solvate, an enantiomer, a diastereoisomer, or a tautomer thereof, or a mixture thereof in any proportion can be prepared into a medicament formulation for external use, oral administration, or injection administration with a pharmaceutically acceptable carrier or excipient.

Therefore, in the present disclosure, the medicament may be an external medication, an oral medication, or an injectable medication.

In the present disclosure, the medicament may contain a pharmaceutically acceptable carrier or excipient. The medicament can be made into various conventional solid dosage forms such as granules, tablets or capsules, liquid dosage forms such as sprays and injections, or semi-solid dosage forms such as creams. In one aspect, the dosage forms of the medicament can be powder, tablet, coated tablet, granule, capsule, solution, emulsion, suspension, injection, spray, aerosol, powder for inhalation, lotion, liniment, ointment, plaster, paste, gel, patch, etc.

In the present disclosure, the term “pharmaceutically acceptable carrier or excipient” includes any and all solvents, cosolvents, dispersion media, coating materials, surfactants, antioxidants, preservatives (such as antibacterial agents, antifungal agents), isotonic agents, absorption delay agents, salts, preservatives, drug stabilizers, binders, excipients, diluents, glidants, granulating agents, disintegrants, thickeners, tackifier, lubricants, anti-caking agents, moisturizers, wetting agents, chelating agents, plasticizers, dyes, flavoring agents, and combinations thereof, which are well known to those skilled in the art (see, for example, Remington's Pharmaceutical Sciences, 19th Ed. Mack Printing Company, 1995; Application Technology of Medicinal Excipients (2nd Ed), edited by Shanghai Pharmaceutical Industry Research Institute, etc, China Medical Science and Technology Press, 2002; Handbook of Comparative Standards for Medicinal Excipients in Various Countries, Volumes 1-3, compiled by the National Pharmacopoeia Committee, China Medical Science and Technology Press, 2016; Handbook of Medicinal Excipients, edited by Raymond C Rowe, Paul J Sheskey, and Paul J Weller, translated by Zheng Jun Min, Chemical Industry Press, 2005, etc). Except for carriers and excipients that are incompatible with the active ingredients, any conventional carriers and excipients may be considered for use in therapeutic or pharmaceutical compositions.

For example, in solid dosage forms, the pharmaceutically acceptable carriers or excipients may include at least one of the following: (a) fillers such as starch, corn starch, modified starch, compressible starch, lactose, lactose monohydrate, microcrystalline cellulose, cyclodextrin, sorbitol, mannitol, calcium phosphate, amino acids, etc; (b) binders, such as starch paste, gelatinized starch, sodium carboxymethyl cellulose, hydroxypropyl cellulose, hydroxypropyl methyl cellulose, methyl cellulose, ethyl cellulose, hydroxypropyl methyl cellulose, low substituted hydroxypropyl cellulose, polyvinylpyrrolidone, gelatin, alginates, etc.; (c) moisturizers, such as glycerin; (d) disintegrants, such as dry starch, modified starch, carboxymethyl starch sodium, low substituted hydroxypropyl cellulose, cross-linked polyvinylpyrrolidone, cross-linked carboxymethyl cellulose sodium, microcrystalline cellulose, effervescent disintegrants, cross-linked polyvinylpyrrolidone, etc.; (e) solution blockers, such as paraffin; (f) absorption enhancers, such as quaternary ammonium compounds; (g) wetting agents, such as cetyl alcohol and glyceryl monostearate; (h) absorbents, such as kaolin and bentonite; (i) lubricants such as talc, stearic acid, magnesium or calcium stearate, micronized silica, hydrogenated castor oil, and solid polyethylene glycol, polyethylene glycol 4000-20000, magnesium dodecyl sulfate, etc.

In the present disclosure, the medicament is applicable to humans or other isothermal animals. When the applicable subject is human, the preferred single or mixed dosage of tropic acid and its derivatives is 1 mg/kg·d to 50 mg/kg·d, and further preferred is 10 mg/kg·d to 20 mg/kg·d. The effective therapeutic amount of a compound or pharmaceutical composition depends on the individual's species, body weight, age, individual condition, the disease being treated or its severity. Physicians, clinical physicians, or veterinarians with ordinary skills can easily determine the effective amount of each active ingredient needed to prevent, treat, or inhibit the progression of diseases.

The present disclosure also provides a compound represented by formula A, a pharmaceutically acceptable salt thereof, a solvate, an enantiomer, a diastereoisomer, a tautomer, or a mixture thereof in any proportion:

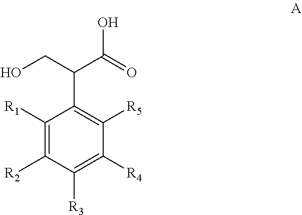

wherein, R₁-R₅ are each independently selected from —H or —OH;
provided that at least two of R₁-R₅ are selected from —OH.

In one embodiment, two or three of R₁-R₅ are selected from —OH.

In one embodiment, the compound is selected from the following compounds represented by formulas III-IV:

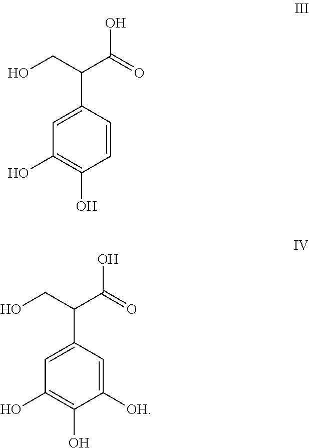 [SMILES: O=C(O)C(CO)c1ccc(O)c(O)c1 ; O=C(O)C(CO)c1cc(O)c(O)c(O)c1]

The pharmaceutically acceptable salts of the compounds of the present disclosure include their base addition salts and acid addition salts. Preferably, the base addition salt is selected from the group consisting of sodium salt, potassium salt, calcium salt, lithium salt, magnesium salt, zinc salt, ammonium salt, tetramethylammonium salt, tetraethylammonium salt, triethylamine salt, trimethylammonium salt, ethylamine salt, diethanolamine salt, arginine salt, or lysine salt; The acid addition salt is selected from organic acid salts such as acetate, aspartate, benzoate, benzenesulfonate, citrate, ethanedisulfonate, ethanesulfonate, formate, fumarate, gluconate, glucuronate, lactate, malate, trifluoroacetate, maleic acid, as well as inorganic acid salts such as hydrochloride, hydrobromide, bisulfate, nitrate, phosphate, etc. The compounds of the present disclosure in free form can be converted into the corresponding compounds in salt form, and vice versa. The compounds of the present disclosure in free or salt forms and solvate forms can be converted into the corresponding compounds in non solvate forms in free or salt forms, and vice versa.

The compound of the present disclosure also includes its solvate form, which refers to the complex formed by one or more solvent molecules with the compound of the present disclosure. The solvents that form solvates include, but are not limited to, water, isopropanol, ethanol, methanol, dimethyl sulfoxide, ethyl acetate, acetic acid, and aminoethanol.

The compounds of the present disclosure can exist in the forms of isomers and mixtures thereof, such as tautomers, optical isomers, enantiomers, and diastereoisomers. The compounds of the present disclosure may contain an asymmetric carbon atom and therefore may exist in the form of enantiomers or diastereoisomers and mixtures thereof, such as racemates. The compounds of the present disclosure can exist in (R)-, (S)-, or (R, S)-configurations, preferably in (R)- or (S)-configurations at specific positions of the compound.

The present disclosure has the following advantages and effects compared to the prior art:
(1) The present disclosure discovered for the first time that tropic acid and its derivatives can significantly alleviate skin damage in animal models of psoriasis;
(2) Compared with existing drugs, tropic acid and its derivatives, as the main ingredients for treating psoriasis, have been developed into drugs that have low toxicity, rapid onset, short treatment course, small dosage, low recurrence rate, and convenient use. They also take external, oral, and injection dosage forms into account, and are suitable for patients with different degrees and types of psoriasis;
(3) Compounds III and IV, obtained through structural modification, exhibit more significant therapeutic effects than tropic acid; and
(4) The small molecule drug used in the present disclosure is easy to obtain, inexpensive, stable in properties, easy to store and transport, and has broad application prospects.

### BRIEF DESCRIPTION OF DRAWINGS

FIG. 1. ¹H-NMR spectrum of compound III;

FIG. 2. ¹³C-NMR spectrum of compound III;

FIG. 3. ¹H-NMR spectrum of compound IV;

FIG. 4. ¹³C-NMR spectrum of compound IV;

FIG. 5A-B. Comparative photos of back skin lesions (day 7) in each group of mice;

FIG. 6. Severity index of skin lesion area (PASI) scores for each group of mice (n=10);

FIG. 7. Histopathological HE staining photos of skin lesions in each group of mice (×200);

FIG. 8. Statistical data of epidermal thickness of mice in each group (n=10).

### DETAILED DESCRIPTION

The preferred examples of the present disclosure are described below in conjunction with the accompanying drawings. It should be understood that the preferred examples described herein are only for illustrating and explaining the present disclosure, and are not intended to limit the present disclosure. The described examples are only a part of the examples of the present disclosure, not all of them. Based on the examples of the present disclosure, all other examples obtained by ordinary skilled persons in the art without creative labor are within the scope of protection of the present disclosure.

### Example 1: Preparation of Compound of Formula III (3,4-dihydroxy-α-(hydroxymethyl)phenylacetic acid)

### 1. Synthesis Process

Under ice water bath conditions, 1.96 g (10 mmol, 1.0 eq.) of 3,4-dimethoxyphenylacetic acid was taken into a 100 mL Schlenk flask. Under nitrogen protection, 20 mL of anhydrous DCM was added, and 1.8 mL (25 mmol, 2.5 eq.) of dichlorosulfoxide was slowly added dropwise while maintaining low temperature. The system was transferred to room temperature and stirred for 2 hours. Solvent and excess thionyl chloride were removed under reduced pressure at 40° C. Subsequently, 20 mL of methanol was added to the system and stirred overnight at room temperature. The solvent was removed by distillation under reduced pressure to obtain 1.50 g of methyl 3,4-dimethoxyphenylacetate.

At room temperature, 1.0 g (4.76 mmol, 1.0 eq.) of compound methyl 3,4-dimethoxyphenylacetate was taken into a 100 mL Schlenk flask, then 0.171 g paraformaldehyde (5.71 mmol, 1.2 eq.) and 5 mL of DMSO were added. Under nitrogen protection, 0.0257 g catalytic amount (0.476 mmol, 10 mol %) of sodium methoxide was added to the system, and the system was stirred overnight at room temperature. After post-treatment, the reaction mixture was transferred to 100 mL of water, the aqueous layer was extracted with 3×20 mL of EtOAc, the organic phases was combined, washed with water and dried, concentrated under reduced pressure, and purified by column separation to obtain 0.90 g of methyl 3,4-dimethoxy-α-(hydroxymethyl)phenylacetate.

0.6 g of methyl 3,4-dimethoxy-α-(hydroxymethyl)phenylacetate (2.5 mmol, 1.0 eq.) was taken into a 20 mL Schlenk flask, and 2 mL of hydrogen bromide solution (40%) was slowly added to the system at room temperature. The reaction was heated and refluxed overnight. The reaction system was transferred to 50 mL of water, the aqueous layer was extracted with 3×20 mL of DCM, the organic phases was combined, washed with water and dried, concentrated under reduced pressure, and purified by column separation to obtain 0.35 g of 3,4-dihydroxy-α-(hydroxymethyl)phenylacetic acid.

### 2. Structural Identification

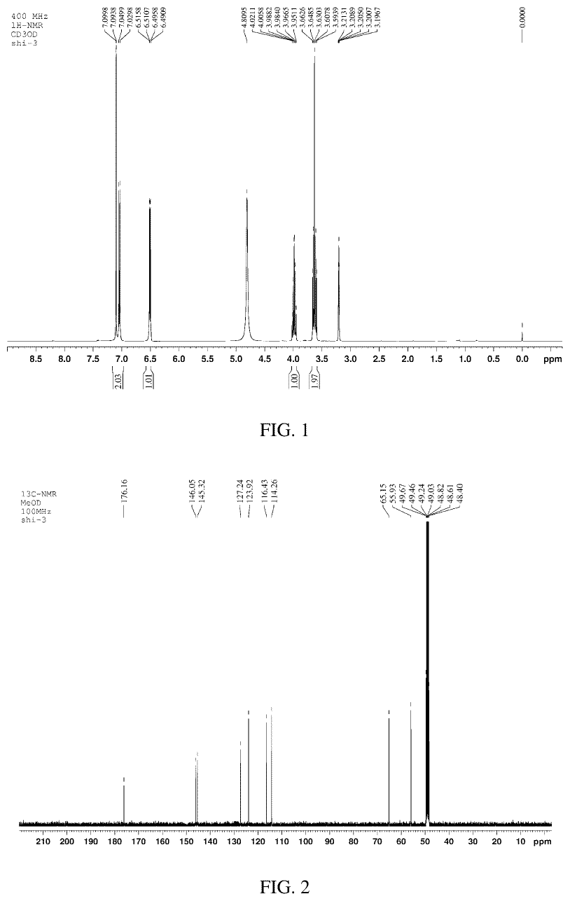

3,4-dihydroxy-α-(hydroxymethyl)phenylacetic acid is a white crystalline powder that is easily soluble in methanol and water. ¹H-NMR (400 MHz, CD₃OD) is shown in FIG. 1, δ_{H} (ppm) 3.63 (2H, m, CH₂), 3.99 (1H, m, CH), 6.50 (1H, dd, J=2.0, 8.0 Hz, Ph-H), 7.04 (1H, d, J=8.0 Hz, Ph-H), 7.09 (1H, d, J=2.0 Hz, Ph-H); ¹³C-NMR (400 MHz, CD₃OD) is shown in FIG. 2, δ_{C} (ppm) 55.9 (CH), 65.2 (CH₂), 114.3 (CH), 116.4 (CH), 123.9 (CH), 127.2 (C), 145.3 (C), 146.0 (C), 176.2 (C═O).

### Example 2: Preparation of Compound of Formula IV (3,4,5-trihydroxy-α-(hydroxymethyl)phenylacetic acid)

### 1. Synthesis Process

Under ice water bath conditions, 2.26 g of 3,4,5-trimethoxyphenylacetic acid (10 mmol, 1.0 eq.) was taken into a 100 mL Schlenk flask. Under nitrogen protection, 20 mL of anhydrous DCM was added, and 1.8 mL of dichlorosulfoxide (25 mmol, 2.5 eq.) was slowly added dropwise while maintaining low temperature. The system was transferred to room temperature and stirred for 2 hours. Solvent and excess thionyl chloride were removed under reduced pressure at 40° C. Subsequently, 20 mL of methanol was added to the system and stirred overnight at room temperature. The solvent was removed by distillation under reduced pressure to obtain 1.65 g of methyl 3,4,5-trimethoxyphenylacetate.

At room temperature, 1.5 g of compound methyl 3,4,5-trimethoxyphenylacetate (6.25 mmol, 1.0 eq.) was taken into a 100 mL Schlenk flask, and 0.224 g of paraformaldehyde (7.50 mmol, 1.2 eq.) and 5 mL of DMSO were added. Under nitrogen protection, 0.0338 g of catalytic amount of sodium methoxide (0.625 mmol, 10 mol %) was added to the system and the system was stirred overnight at room temperature while maintaining room temperature. After post-treatment, the reaction was transferred to 100 mL of water, and the aqueous layer was extracted with 3×20 mL of EtOAc. After the organic phases were combined, the mixture was washed with water, dried, and concentrated under reduced pressure before column separation and purification to obtain 1.17 g of methyl 3,4,5-trimethoxy-α(hydroxymethyl)phenylacetate.

1.0 g of methyl 3,4,5-trimethoxy-α-(hydroxymethyl)phenylacetate (3.70 mmol, 1.0 eq.) was taken into a 20 mL Schlenk flask, and 2 mL of hydrogen bromide solution (40%) was slowly added to the system at room temperature. The reaction was heated and refluxed overnight. After post-treatment, the reaction system was transferred to 50 mL of water, the aqueous layer was extracted with 3×20 mL of DCM, the organic phases were combined, washed with water and dried, concentrated under reduced pressure, and purified by column separation to obtain 0.69 g of 3,4,5-trihydroxy-α-(hydroxymethyl)phenylacetic acid.

### 2. Structural Identification

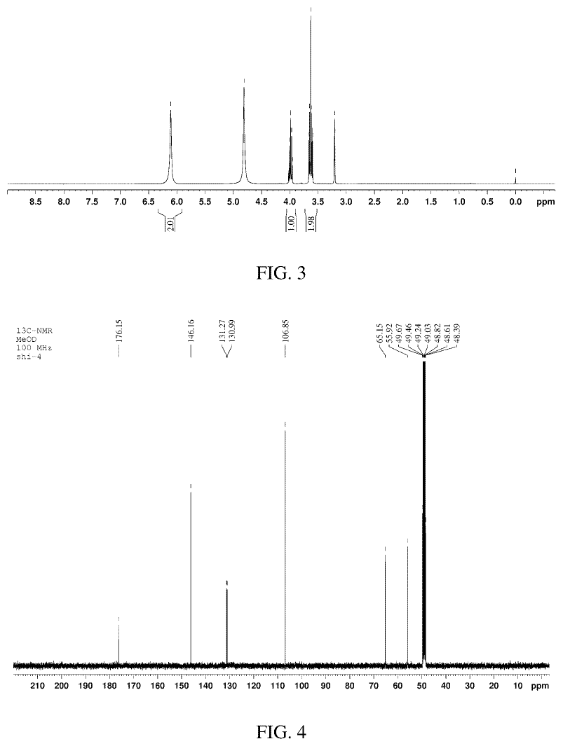

3,4-dihydroxy-α-(hydroxymethyl)phenylacetic acid is a white crystalline powder that is easily soluble in methanol and water. ¹H-NMR (400 MHz, CD₃OD) is shown in FIG. 3, (H (ppm) 3.63 (2H, m, CH₂), 3.99 (1H, m, CH), 6.10 (2H, s, Ph-H); ¹³C-NMR (400 MHz, CD₃OD) is shown in FIG. 4, δ_{C} (ppm) 55.9 (CH), 65.2 (CH₂), 106.8 (2×CH), 131.0 (C), 131.3 (C), 146.2 (2×C), 176.2 (C═O).

### Example 3: Study on the Effect of Tropic Acid and its Derivatives on Psoriasis in Mice Induced by Imiquimod

### 1. Animal Modeling, Grouping, and Administration

110 male Balb/c mice (8-10 weeks, 20-25 g each) were anesthetized by intraperitoneal injection of 10% chloral hydrate, and approximately 2 cm×4 cm of back hair was shaved off. The mice were randomly divided into 11 groups, with 10 mice in each group, including a blank control group, a model group, a positive drug group, a high-dose tropic acid group (A), a low-dose tropic acid group (B), a high-dose 4-hydroxy-α-(hydroxymethyl)phenylacetic acid group (C), a low-dose 4-hydroxy-α-(hydroxymethyl)phenylacetic acid group (D), a high-dose 3,4-dihydroxy-α-(hydroxymethyl)phenylacetic acid group (E), a low-dose 3,4-dihydroxy-α-(hydroxymethyl)phenylacetic acid group (F), a high-dose 3,4,5-trihydroxy-α-(hydroxymethyl)phenylacetic acid group (G), and a low-dose 3,4,5-trihydroxy-α-(hydroxymethyl)phenylacetic acid group (H) of. Except for the blank control group, which received no treatment, all other groups were treated with 50 mg of 5% imiquimod cream daily to prepare a psoriasis mouse model. All groups were administered simultaneously, with dosing twice daily, and the grouping and dosing regimen are detailed in Table 1. The treatment was continued for 6 days. On the 7th day, the skin lesions of the mice were photographed and recorded, after which the mice were euthanized and their back skin was taken for further use.

TABLE 1
Animal grouping and administration information table (n = 10)

| Groups | Daily dosing regimen |
|---|---|
| Blank control | 0.2 mL of normal saline administered by gavage |
| Model | 5% Imiquimod cream 50 mg + 0.2 mL of normal saline administered by gavage |
| Positive drug | 5% Imiquimod cream 50 mg + dexamethasone 5 mg/kg · d administered by gavage |
| A | 5% Imiquimod cream 50 mg + r tropic acid 6 mg/kg · d administered by gavage |
| B | 5% Imiquimod cream 50 mg + tropic acid 3 mg/kg · d administered by gavage |
| C | 5% Imiquimod cream 50 mg + 4-hydroxy-α-(hydroxymethyl)phenylacetic acid 6 |
| | mg/kg · d administered by gavage |
| D | 5% Imiquimod cream 50 mg + 4-hydroxy-α-(hydroxymethyl)phenylacetic acid 3 |
| | mg/kg · d administered by gavage |
| E | 5% Imiquimod cream 50 mg + 3,4-dihydroxy-α-(hydroxymethyl)phenylacetic acid 6 |
| | mg/kg · d administered by gavage |
| F | 5% Imiquimod cream 50 mg + 3,4-dihydroxy-α-(hydroxymethyl)phenylacetic acid 3 |
| | mg/kg · d administered by gavage |
| G | 5% Imiquimod cream 50 mg + 3,4,5-trihydroxy-α-(hydroxymethyl)phenylacetic acid 6 |
| | mg/kg · d administered by gavage |
| H | 5% Imiquimod cream 50 mg + 3,4,5-trihydroxy-α-(hydroxymethyl)phenylacetic acid 3 |
| | mg/kg · d administered by gavage |

### 2. Indices Detection and Observation

Histomorphological observation: Starting from the first day of modeling, the degree of erythema, scales, and inflammatory infiltration thickening on the back of mice were scored daily according to the PASI scoring criteria. The PAIS scoring criteria were as follows: 0-none; 1-mild; 2-moderate; 3-severe cases; 4-extremely severe. The more severe the degree of skin damage, the higher the score. The three points were summed to obtain the total PASI score. The average score of each group of mice were then plotted to create a trend line to observe the changes in skin lesions of each group of mice. Digital photography was used to record the skin lesions on the back of the mice in each group on the 7th day for observation and comparison.

Histopathological examination: On the 7th day of modeling, the mice were euthanized, and the back skin of each group of mice was cut and fixed in 4% paraformaldehyde for 24 hours, embedded in paraffin, and sliced into 4 μm sections. HE staining, sealing, and observation of histopathological changes under microscope were performed, and the epidermal thickness was measured using the scale function of the cellSens standard.

### 3. Experimental Results and Discussion

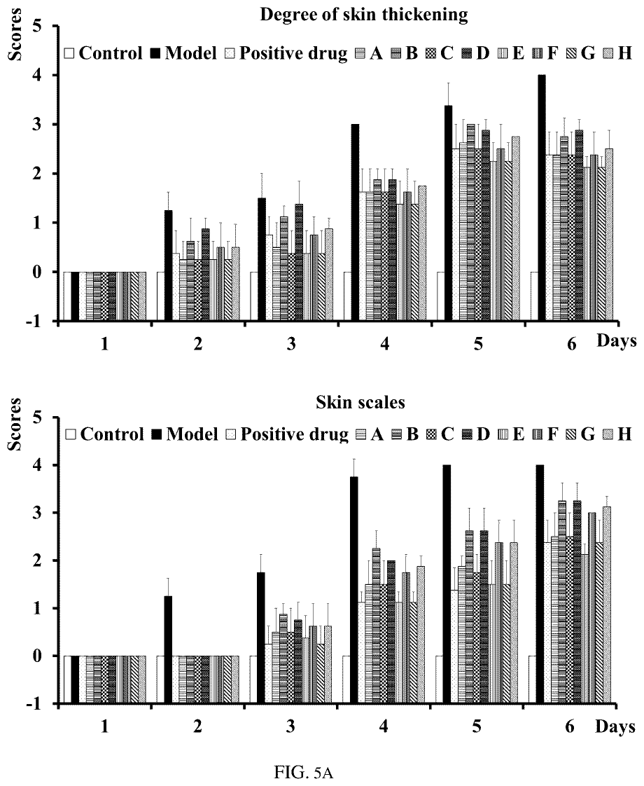

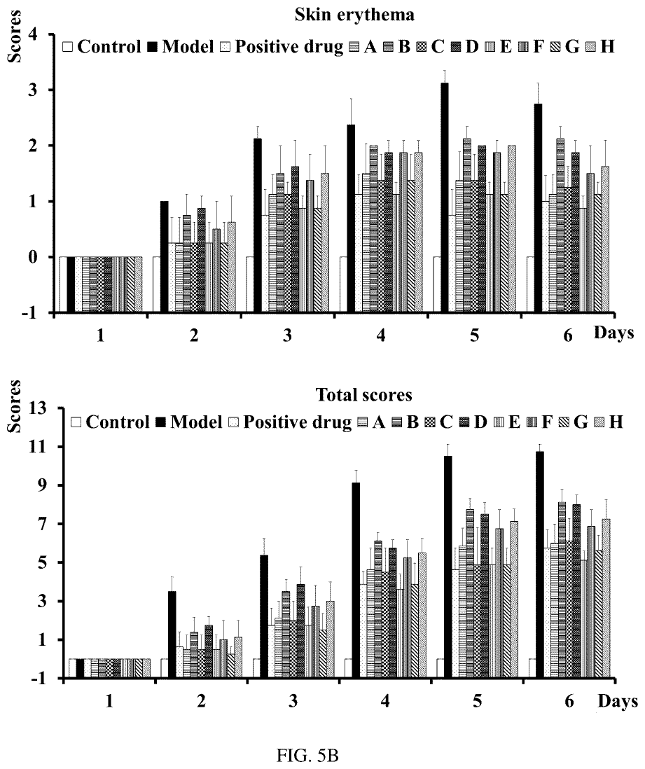

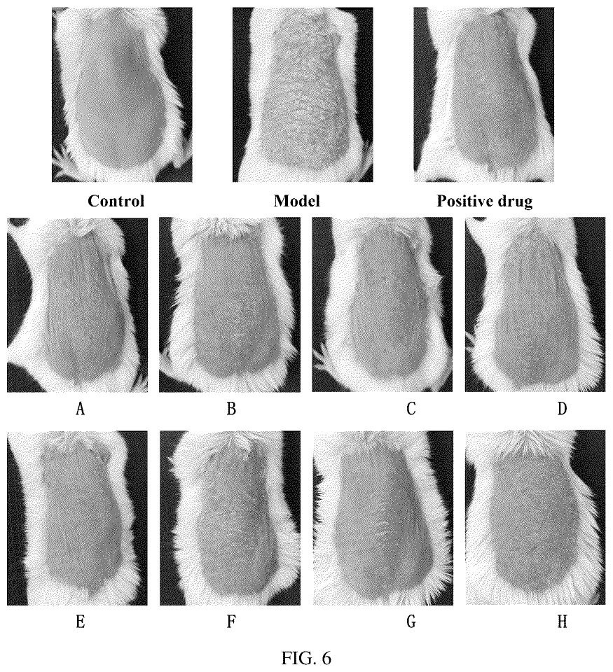

Histomorphological observation: Changes in the back skin of mice were observed daily. The skin of the blank control group mice was smooth and undamaged. According to the PASI score, compared with the blank control group, the skin of the model group mice began to show significant damage on the second day, and the degree of skin lesions in the mice increased sharply from day 4 to 6 (P<0.01). The skin of the mice had obvious erythema and a sense of substance when pressed. The skin lesions were covered with thick yellow-white scales, and there was obvious inflammatory infiltration and thickening of the skin. During the treatment with tropic acid and its derivatives, the degree of skin lesions in mice of both the high-dose and low-dose groups was significantly reduced, and all the scores were significantly improved. Among them, the high-dose group showed the most significant improvement after 3-6 days (P<0.01), as shown in FIGS. 5A and 5B. On the 7th day, compared with the model group, the skin scales of the mice in each group treated with tropic acid and its derivatives were reduced, the skin was smoother, and the degree of skin damage was significantly reduced. This indicates that tropic acid and its derivatives treatment can improve the psoriasis-like skin damage induced by imiquimod in mice, as shown in FIG. 6.

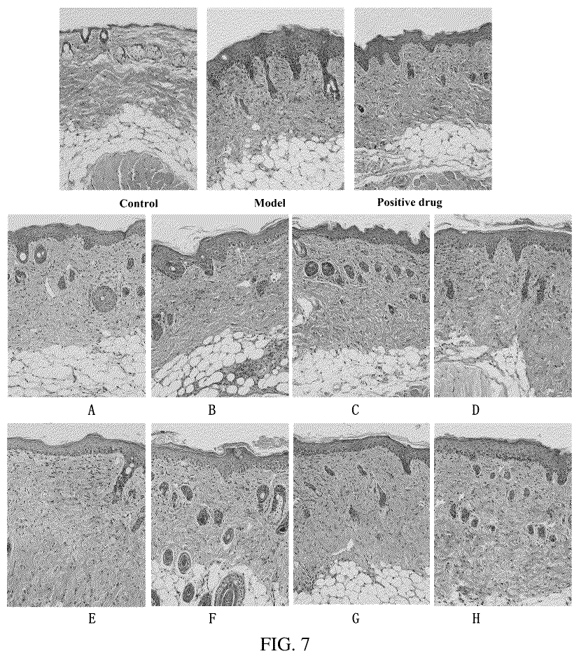

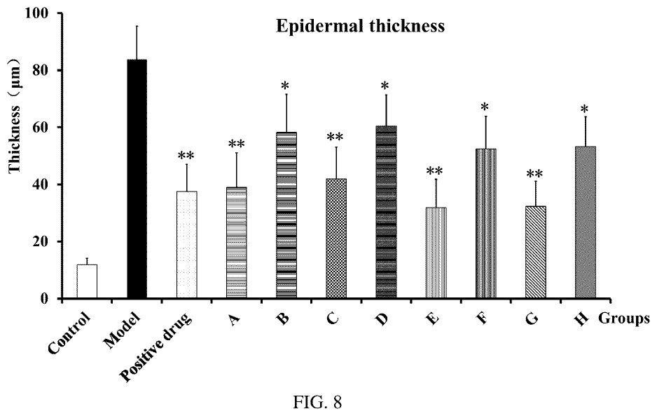

Histopathological examination: As shown in HE staining, compared with the control group, the thickness of the stratum spinosum in the skin tissue of the model group mice was increased significantly, with wider intercellular spaces, indicating the presence of intercellular edema. A large number of incompletely keratinized cells were observed in the outermost stratum corneum of the epidermis, showing histopathological features similar to those of human psoriasis. Compared with the model group, the thickness of the stratum spinosum was significantly reduced in the positive drug group and the each group treated with tropic acid and its derivatives, with significantly improvements in incomplete skin keratinization. Among them, the improvement was more significant at high dose groups, as shown in FIG. 7. The thickness of the epidermal layer was measured, and the results showed that compared with the control group, the epidermal thickness of the model group mice significantly increased (P<0.01). Compared with the model group, the epidermal thickness of mice in the groups treated with tropic acid and its derivatives was significantly reduced, and the epidermal thickness of mice in the high-dose groups of tropic acid and its derivatives was more significantly reduced (P<0.01), as shown in FIG. 8.

In addition, after 6 days of treatment, compared with the high-dose group of tropic acid, the high-dose groups of compound III and compound IV showed a significant decrease in the degree of inflammatory infiltration and thickening of the skin in mice, and the difference was statistically significant (P<0.05), as shown in FIG. 5A. After 6 days of treatment, the degree of scale reduction in mice treated with high-dose compound III and compound IV was superior to that of mice treated with high-dose tropic acid, and the difference was statistically significant (P<0.05), as shown in FIG. 5A. After 6 days of treatment, compared with the high-dose group of tropic acid, the skin erythema score of mice in the high-dose group of compound III was significantly reduced, and the difference was statistically significant (P<0.05). Compared with the low-dose group of tropic acid, the skin erythema score of mice in the low-dose groups of compound III and compound IV was significantly reduced, and the difference was statistically significant (P<0.01), as shown in FIG. 5B. In terms of overall score, the skin lesion scores of mice in the high-dose and low-dose groups of compound III and compound IV were superior to those of mice in the same dose group of tropic acid, and the difference was statistically significant (high-dose compound III group vs high-dose tropic acid group, P<0.01; high-dose compound IV group vs high-dose tropic acid group, P<0.05; the low-dose groups of compounds III and IV compared to the low-dose tropic acid group, P<0.05), As shown in FIG. 5B.

The above results indicate that tropic acid and its derivatives have significant therapeutic effects on the back skin injury induced by imiquimod in a mouse model of psoriasis, with compounds III and IV showing the best therapeutic effect.

### Example 4: Preparations of Tropic Acid and its Derivatives Tablets

The formulas are shown in Table 2. According to formula 1, the active pharmaceutical ingredient (tropic acid) and excipients (lactose, microcrystalline cellulose, polyvinylpyrrolidone, croscarmellose sodium, micronized silica, magnesium stearate, and purified water) were taken. Tropic acid, lactose, microcrystalline cellulose, and polyvinylpyrrolidone were added to a wet granulating machine, and purified water was used as a wetting agent for wet granulation. After wet granulation, drying, and dry granulation, dry granules were obtained. Croscarmellose sodium, micronized silica, and magnesium stearate were added to the dry granules and mixed evenly. The well-mixed materials were then compressed using a tablet press to obtain tropic acid tablets. The active pharmaceutical ingredients and excipients from formulas 2, 3, and 4 were taken respectively and prepared following the same process as described above to produce 4-hydroxy-α-(hydroxymethyl)phenylacetic acid tablets, 3,4-dihydroxy-α-(hydroxymethyl)phenylacetic acid tablets, and 3,4,5-trihydroxy-α-(hydroxymethyl)phenylacetic acid tablets

TABLE 2
formulas of tropic acid and its derivatives tablets

| | formula | formula | formula | formula |
|---|---|---|---|---|
| Name of materials | 1 (g) | 2 (g) | 3 (g) | 4 (g) |
| tropic acid | 200 | — | — | — |
| 4-hydroxy-α- | — | 200 | — | — |
| (hydroxymethyl) | | | | |
| phenylacetic acid | | | | |
| 3,4-dihydroxy-α- | — | — | 200 | — |
| (hydroxymethyl) | | | | |
| phenylacetic acid | | | | |
| 3,4,5-trihydroxy-α- | — | — | — | 200 |
| (hydroxymethyl) | | | | |
| phenylacetic acid | | | | |
| lactose | 980 | 980 | 980 | 980 |
| microcrystalline | 700 | 700 | 700 | 700 |
| cellulose | | | | |
| polyvinylpyrrolidone | 160 | 160 | 160 | 160 |
| croscarmellose | 80 | 80 | 80 | 80 |
| sodium | | | | |
| micronized silica | 20 | 20 | 20 | 20 |
| magnesium stearate | 20 | 20 | 20 | 20 |
| purified water | 340 | 340 | 340 | 340 |

### Example 5: Preparations of Coated Tablets of Tropic Acid and its Derivatives

The formulas are shown in Table 3. According to formula 5, the active pharmaceutical ingredient (tropic acid) and excipients (lactose, microcrystalline cellulose, hydroxypropyl methylcellulose, sodium dodecyl sulfate, sodium carboxymethyl starch, micronized silica, magnesium stearate, film coating premix, and purified water) were taken. Sodium dodecyl sulfate was added to purified water and stirred to dissolution. Tropic acid, lactose, microcrystalline cellulose, and hydroxypropyl methylcellulose were added to a wet granulating machine, and an aqueous solution of sodium dodecyl sulfate was used as a wetting agent for wet granulation. After wet granulation, drying, and dry granulation, dry granules were obtained. Sodium carboxymethyl starch, micronized silica, and magnesium stearate were added to the dry granules and mixed evenly. The well-mixed materials were then compressed using a tablet press to obtain the tropic acid tablets. The film coating premix was added to purified water and stirred continuously for more than 1 hour to obtain the film coating solution. Then, the prepared tablets were coated in the coating machine to obtain the tropic acid coated tablets. The active pharmaceutical ingredients and excipients from formulas 6, 7, and 8 were taken respectively, and prepared following the same process as described above to produce 4-hydroxy-α-(hydroxymethyl)phenylacetic acid coated tablets, 3,4-dihydroxy-α-(hydroxymethyl)phenylacetic acid coated tablets, and 3,4,5-trihydroxy-α-(hydroxymethyl)phenylacetic acid coated tablets.

TABLE 3
formulas of tropic acid and its derivativescoated tablets

| | formula | formula | formula | formula |
|---|---|---|---|---|
| Name of materials | 5 (g) | 6 (g) | 7 (g) | 8 (g) |
| tropic acid | 100 | — | — | — |
| 4-hydroxy-α- | — | 100 | — | — |
| (hydroxymethyl)phenylacetic | | | | |
| acid | | | | |
| 3,4-dihydroxy-α- | — | — | 100 | — |
| (hydroxymethyl)phenylacetic | | | | |
| acid | | | | |
| 3,4,5-trihydroxy-α- | — | — | — | 100 |
| (hydroxymethyl)phenylacetic | | | | |
| acid | | | | |
| lactose | 960 | 960 | 960 | 960 |
| microcrystalline cellulose | 600 | 600 | 600 | 600 |
| hydroxypropyl methylcellulose | 200 | 200 | 200 | 200 |
| sodium dodecyl sulfate | 20 | 20 | 20 | 20 |
| sodium carboxymethyl starch | 80 | 80 | 80 | 80 |
| micronized silica | 20 | 20 | 20 | 20 |
| magnesium stearate | 20 | 20 | 20 | 20 |
| purified water | 320 | 320 | 320 | 320 |
| film coating premix | 80 | 80 | 80 | 80 |
| purified water (for film | 667 | 667 | 667 | 667 |
| coating) | | | | |

### Example 6: Preparations of Tropic Acid and its Derivatives Spray I

The prescriptions are shown in Table 4. According to formula 9, the active pharmaceutical ingredient (tropic acid) and excipients (polyvinylpyrrolidone K30, propylene glycol monocaprylate, ethylparaben, poloxamer, di-tert-butyl-p-cresol (BHT), 1N sodium hydroxide solution, ethanol, and water) were taken. Active pharmaceutical ingredient, polyvinylpyrrolidone K30, propylene glycol monocaprylate, ethylparaben, and BHT were added to ethanol and stirred until completely dissolved. Poloxamer was added in an appropriate amount of water for dissolution; then the poloxamer aqueous solution was added to the alcohol solution of the above mixture, and the pH value was adjusted to the range of 3-8 with 1N sodium hydroxide solution. Water was added to 200 mL to obtain a spray solution, which was filled into a spray bottle to obtain tropic acid spray I. The active pharmaceutical ingredients and excipients of formulas 10, 11 and 12 were taken respectively, and prepared following the same process as described above to produce 4-hydroxy-α-(hydroxymethyl)phenylacetic acid spray I, 3,4-dihydroxy-α-(hydroxymethyl)phenylacetic acid spray I and 3,4,5-trihydroxy-α-(hydroxymethyl)phenylacetic acid spray I.

TABLE 4
formulas of tropic acid and its derivatives spray I

| | formula 9 | formula 10 | formula 11 | formula 12 |
|---|---|---|---|---|
| Name of materials | (g) | (g) | (g) | (g) |
| tropic acid | 4.0 g | — | — | — |
| 4-hydroxy-α- | — | 4.0 g | — | — |
| (hydroxymethyl)phenylacetic acid | | | | |
| 3,4-dihydroxy-α- | — | — | 4.0 g | — |
| (hydroxymethyl)phenylacetic acid | | | | |
| 3,4,5-trihydroxy-α- | — | — | — | 4.0 g |
| (hydroxymethyl)phenylacetic acid | | | | |
| polyvinylpyrrolidone K30 | 8.0 g | 8.0 g | 8.0 g | 8.0 g |
| propylene glycol monocaprylate | 15.0 g | 15.0 g | 15.0 g | 15.0 g |
| ethylparaben | 0.2 g | 0.2 g | 0.2 g | 0.2 g |
| poloxamer | 1.0 g | 1.0 g | 1.0 g | 1.0 g |
| BHT | 0.5 g | 0.5 g | 0.5 g | 0.5 g |
| 1N sodium hydroxide solution | appropriate | appropriate | appropriate | appropriate |
| | amount | amount | amount | amount |
| ethanol | 80 g | 80 g | 80 g | 80 g |
| water | added to 200 | added to 200 | added to 200 | added to 200 |
| | mL | mL | mL | mL |

### Example 7: Preparations of Tropic Acid and its Derivatives Spray II

The prescriptions are shown in Table 5. According to formula 13, the active pharmaceutical ingredient (tropic acid) and excipients (hydroxypropyl cellulose, propylene glycol monocaprylate, ethylparaben, ethanol, and water) were taken, and active pharmaceutical ingredient, hydroxypropyl cellulose, propylene glycol monocaprylate, and ethylparaben were added to ethanol, stirred until dissolved completely. Water was added to 200 mL to obtain the spray solution. The solution was filled into a spray bottle to obtain tropic acid spray II. The active pharmaceutical ingredients and excipients of formulas 14, 15 and 16 were taken respectively, and prepared following the same process as described above to produce 4-hydroxy-α-(hydroxymethyl)phenylacetic acid spray II, 3,4-dihydroxy-α-(hydroxymethyl)phenylacetic acid spray II and 3,4,5-trihydroxy-α-(hydroxymethyl)phenylacetic acid spray II.

TABLE 5
formulas of tropic acid and its derivatives spray II

| Name of materials | formula 13 (g) | | formula 14 (g) | | formula 15 (g) | | formula 16 (g) | |
|---|---|---|---|---|---|---|---|---|
| tropic acid | 2.0 | g | — | | — | | — | |
| 4-hydroxy-α- | — | | 2.0 | g | — | | — | |
| (hydroxymethyl)phenylacetic acid | | | | | | | | |
| 3,4-dihydroxy-α- | — | | — | | 2.0 | g | — | |
| (hydroxymethyl)phenylacetic acid | | | | | | | | |
| 3,4,5-trihydroxy-α- | — | | — | | — | | 2.0 | g |
| (hydroxymethyl)phenylacetic acid | | | | | | | | |
| hydroxypropyl cellulose | 6 | g | 6 | g | 6 | g | 6 | g |
| propylene glycol monocaprylate | 20 | g | 20 | g | 20 | g | 20 | g |
| ethylparaben | 0.2 | g | 0.2 | g | 0.2 | g | 0.2 | g |
| ethanol | 100 | g | 100 | g | 100 | g | 100 | g |

| | | | | |
|---|---|---|---|---|
| water | added to 200 | added to 200 | added to 200 | added to 200 |
| | mL | mL | mL | mL |

### Example 8: Preparation of Tropic Acid and its Derivatives Injection I

The formulas are shown in Table 6. According to formula 17, the active pharmaceutical ingredient (tropic acid) and excipients (sodium chloride, disodium hydrogen phosphate, sodium dihydrogen phosphate, poloxamer, sodium bisulfite, and water for injection) were taken, the active pharmaceutical ingredient and poloxamer were added to water for injection for dissolution, then sodium chloride, disodium hydrogen phosphate, sodium dihydrogen phosphate, and sodium bisulfite were added to the above solution for dissolution, and finally water was added to 100 mL to obtain the injection solution. The above injection solution was filled into ampoules or penicillin vials of the corresponding volume to obtain tropic acid injection I. The active pharmaceutical ingredient and excipients from formulas 18, 19, and 20 were taken respectively, and prepared following the same process as described above to produce 4-hydroxy-α-(hydroxymethyl)phenylacetic acid injection I, 3,4-dihydroxy-α-(hydroxymethyl)phenylacetic acid injection I, and 3,4,5-trihydroxy-α-(hydroxymethyl)phenylacetic acid injection I.

TABLE 6
The formulas of tropic acid and its derivatives injection I

| | formula 17 | | formula 18 | | formula 19 | | formula 20 | |
|---|---|---|---|---|---|---|---|---|
| Name of materials | (g) | | (g) | | (g) | | (g) | |
| tropic acid | 1.0 | g | — | | — | | — | |
| 4-hydroxy-α- | — | | 1.0 | g | — | | — | |
| (hydroxymethyl)phenylacetic acid | | | | | | | | |
| 3,4-dihydroxy-α- | — | | — | | 1.0 | g | — | |
| (hydroxymethyl)phenylacetic acid | | | | | | | | |
| 3,4,5-trihydroxy-α- | — | | — | | — | | 1.0 | g |
| (hydroxymethyl)phenylacetic acid | | | | | | | | |
| sodium chloride | 0.85 | g | 0.85 | g | 0.85 | g | 0.85 | g |
| disodium hydrogen phosphate | 0.21 | g | 0.21 | g | 0.21 | g | 0.21 | g |
| sodium dihydrogen phosphate | 1.6 | mg | 1.6 | mg | 1.6 | mg | 1.6 | mg |
| poloxamer | 0.5 | g | 0.5 | g | 0.5 | g | 0.5 | g |
| sodium bisulfite | 0.1 | g | 0.1 | g | 0.1 | g | 0.1 | g |

| | | | | |
|---|---|---|---|---|
| water for injection | added to 100 | added to 100 | added to 100 | added to 100 |
| | mL | mL | mL | mL |

### Example 9: Preparation of Tropic Acid an its Derivatives Injection II

The prescription is shown in Table 7. According to formula 21, the active pharmaceutical ingredient (tropic acid) and excipients (sodium chloride, disodium hydrogen phosphate, sodium dihydrogen phosphate, Tween 80, water for injection) were taken, the active pharmaceutical ingredient and Tween 80 were added in water for injection, then sodium chloride, disodium hydrogen phosphate, and sodium dihydrogen phosphate were added to the above solution for dissolution, and finally water was added to 100 mL to obtain the injection solution. The above injection solution was filled into ampoules or penicillin vials of the corresponding volume to obtain tropic acid injection II. The active pharmaceutical ingredient and excipients from formulas 22, 23, and 24 were taken, and prepared following the same process as described above to produce 4-hydroxy-α-(hydroxymethyl)phenylacetic acid injection II, 3,4-dihydroxy-α-(hydroxymethyl)phenylacetic acid injection II, and 3,4,5-trihydroxy-α-(hydroxymethyl)phenylacetic acid injection II.

TABLE 7
The formulas of tropic acid and its derivatives injection II

| | formula 21 | | formula 22 | | formula 23 | | formula 24 | |
|---|---|---|---|---|---|---|---|---|
| Name of materials | (g) | | (g) | | (g) | | (g) | |
| tropic acid | 0.5 | g | — | | — | | — | |
| 4-hydroxy-α- | — | | 0.5 | g | — | | — | |
| (hydroxymethyl)phenylacetic acid | | | | | | | | |
| 3,4-dihydroxy-α- | — | | — | | 0.5 | g | — | |
| (hydroxymethyl)phenylacetic acid | | | | | | | | |
| 3,4,5-trihydroxy-α- | — | | — | | — | | 0.5 | g |
| (hydroxymethyl)phenylacetic acid | | | | | | | | |
| sodium chloride | 0.85 | g | 0.85 | g | 0.85 | g | 0.85 | g |
| disodium hydrogen phosphate | 0.21 | g | 0.21 | g | 0.21 | g | 0.21 | g |
| sodium dihydrogen phosphate | 1.6 | mg | 1.6 | mg | 1.6 | mg | 1.6 | mg |
| Tween 80 | 0.3 | g | 0.3 | g | 0.3 | g | 0.3 | g |

| | | | | |
|---|---|---|---|---|
| water for injection | added to 100 | added to 100 | added to 100 | added to 100 |
| | mL | mL | mL | mL |

### Example 10: Preparation of Tropic Acid and its Derivatives Cream I

The formulas are shown in Table 8. According to formula 25, the active pharmaceutical ingredient (tropic acid) and excipients (white vaseline, hexadecanol, Tween 80, diethylene glycol monoethyl ether, ethylparaben, BHT, propylene glycol, citric acid/sodium citrate, and water) were taken. The preparation process was as follows: (1) Dissolution the active pharmaceutical ingredient: propylene glycol was weighed, the active pharmaceutical ingredient was added, stirred and dissolved at 40-50° C.; (2) Oil phase preparation: white vaseline, hexadecanol, ethylparaben, and BHT were weighed, heated to 60-80° C., stirred and dissolved, the dissolved active pharmaceutical ingredient was added slowly, continued stirring, and mixed evenly for later use; (3) Preparation of aqueous phase: purified water was weighed, Tween 80 and diethylene glycol monoethyl ether were added, heated to 60-80° C., and the pH value was adjusted with citric acid/sodium citrate, the solution was stirred and dissolved for later use; (4) Emulsification: the aqueous phase was added slowly to the oil phase, while maintaining at 70° C., the mixture was homogenized, and stirring was continued for more than 30 minutes; (5) Cream formation: the mixture was cooled down, heating was stopped, stirring was continued, and the temperature was gradually reduced to room temperature and the mixture was cooled to form cream, then filled to obtain tropic acid cream I. The active pharmaceutical ingredient and excipients from formulas 26, 27, and 28 were taken respectively, and prepared following the same process as described above to produce 4-hydroxy-α-(hydroxymethyl)phenylacetic acid cream I, 3,4-dihydroxy-α-(hydroxymethyl)phenylacetic acid cream I, and 3,4,5-trihydroxy-α-(hydroxymethyl)phenylacetic acid cream I.

TABLE 8
The formulas of tropic acid and its derivatives cream I

| | formula 25 | | formula 26 | | formula 27 | | formula 28 | |
|---|---|---|---|---|---|---|---|---|
| Name of materials | (g) | | (g) | | (g) | | (g) | |
| tropic acid | 4 | g | — | | — | | — | |
| 4-hydroxy-α-(hydroxymethyl)phenylacetic | — | | 4 | g | — | | — | |
| acid | | | | | | | | |
| 3,4-dihydroxy-α- | — | | — | | 4 | g | — | |
| (hydroxymethyl)phenylacetic acid | | | | | | | | |
| 3,4,5-trihydroxy-α- | — | | — | | — | | 4 | g |
| (hydroxymethyl)phenylacetic acid | | | | | | | | |
| white vaseline | 20 | g | 20 | g | 20 | g | 20 | g |
| hexadecanol | 40 | g | 40 | g | 40 | g | 40 | g |
| Tween 80 | 10 | g | 10 | g | 10 | g | 10 | g |
| diethylene glycol monoethyl ether | 20 | g | 20 | g | 20 | g | 20 | g |
| ethylparaben | 0.4 | g | 0.4 | g | 0.4 | g | 0.4 | g |
| BHT | 0.4 | g | 0.4 | g | 0.4 | g | 0.4 | g |
| propylene glycol | 20 | g | 20 | g | 20 | g | 20 | g |

| | | | | |
|---|---|---|---|---|
| citric acid/sodium citrate | appropriate | appropriate | appropriate | appropriate |
| | amount | amount | amount | amount |
| water | added to 200 g | | | |

### Example 11: Preparation of Tropic Acid and its Derivatives Cream II

The formulas are shown in Table 9. According to formula 29, the active pharmaceutical ingredient (tropic acid) and excipients (white Vaseline, hexadecanol, poloxamer 407, propylene glycol monooctanoate, sodium benzoate, BHA, propylene glycol, glacial acetic acid/sodium acetate, and water) were taken. The preparation process was as follows: (1) Dissolution of the active pharmaceutical ingredient: propylene glycol was weighed, the active pharmaceutical ingredient was added, stirred and dissolved at 40-50° C.; (2) Oil phase preparation: white vaseline, hexadecanol, propylene glycol monooctanoate, and BHA were weighed, heated to 60-80° C., stirred and dissolved, the dissolved active pharmaceutical ingredient was added slowly, stirring was continued, the solution was mixed evenly for later use; (3) Preparation of aqueous phase: purified water was weighed, poloxamer 407 and sodium benzoate were added, the temperature was raised to 60-80° C., the pH value was adjusted with glacial acetic acid/sodium acetate, stirred and dissolved for later use; (4) Emulsification: the aqueous phase was added slowly to the oil phase, the temperature was maintained at 70° C., the mixture was homogenized, and stirring was continued for more than 30 minutes; (5) Cream formation: the mixture was cooled down, heating was stopped, stirring was continued, and the temperature was gradually reduced to room temperature and the mixture was cooled to form paste, then filled to obtain tropic acid cream II. The active pharmaceutical ingredient and excipients from formulas 30, 31, and 32 were taken, and prepared following the same process as described above to produce 4-hydroxy-α-(hydroxymethyl)phenylacetic acid cream I, 3,4-dihydroxy-α-(hydroxymethyl)phenylacetic acid cream I, and 3,4,5-trihydroxy-α-(hydroxymethyl)phenylacetic acid cream II.

TABLE 9
The formulas of tropic acid and its derivatives cream II

| | formula 29 | | formula 30 | | formula 31 | | formula 32 | |
|---|---|---|---|---|---|---|---|---|
| Name of materials | (g) | | (g) | | (g) | | (g) | |
| tropic acid | 8 | g | — | | — | | — | |
| 4-hydroxy-α-(hydroxymethyl)phenylacetic | — | | 8 | g | — | | — | |
| acid | | | | | | | | |
| 3,4-dihydroxy-α- | — | | — | | 8 | g | — | |
| (hydroxymethyl)phenylacetic acid | | | | | | | | |
| 3,4,5-trihydroxy-α- | — | | — | | — | | 8 | g |
| (hydroxymethyl)phenylacetic acid | | | | | | | | |
| white vaseline | 20 | g | 20 | g | 20 | g | 20 | g |
| hexadecanol | 40 | g | 40 | g | 40 | g | 40 | g |
| poloxamer 407 | 10 | g | 10 | g | 10 | g | 10 | g |
| propylene glycol monooctanoate | 20 | g | 20 | g | 20 | g | 20 | g |
| sodium benzoate | 1 | g | 1 | g | 1 | g | 1 | g |
| BHA | 0.4 | g | 0.4 | g | 0.4 | g | 0.4 | g |
| propylene glycol | 20 | g | 20 | g | 20 | g | 20 | g |

| | | | | |
|---|---|---|---|---|
| glacial acetic acid/sodium acetate | appropriate | appropriate | appropriate | appropriate |
| | amount | amount | amount | amount |
| water | added to 200 | added to 200 | added to 200 | added to 200 |
| | g | g | g | g |

The preferred embodiments of the present disclosure were described above, but they are not intended to limit the present disclosure. Those skilled in the art can make improvements and changes to the embodiments disclosed herein without departing from the scope and spirit of the present disclosure.

1. A method of preventing and/or treating psoriasis, comprising administering tropic acid or a derivative thereof, or a pharmaceutically acceptable salt, a solvate, an enantiomer, a diastereoisomer, or a tautomer thereof, or a mixture thereof in any proportion to a subject in need thereof, wherein tropic acid or a derivative thereof has a structure shown in formula A: 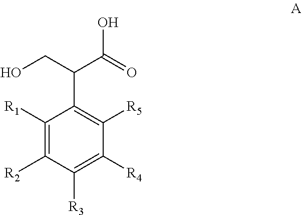
wherein, R₁-R₅ are each independently selected from the group consisting of —H, —OH or a C1-C6 alkoxy.

2. The method according to claim 1, wherein R₁-R₅ are each independently selected from the group consisting of —H or —OH.

3. The method according to claim 1, wherein the tropic acid and a derivative thereof are selected from the following compounds represented by formulas I to IV:

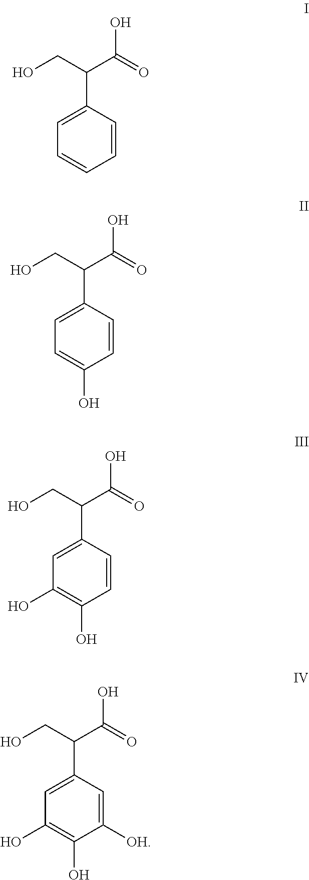 [SMILES: O=C(O)C(CO)c1ccccc1 ; O=C(O)C(CO)c1ccc(O)cc1 ; O=C(O)C(CO)c1ccc(O)c(O)c1 ; O=C(O)C(CO)c1cc(O)c(O)c(O)c1]

4. A medicament for preventing and/or treating psoriasis, comprising the tropic acid or a derivative thereof, or a pharmaceutically acceptable salt, a solvate, an enantiomer, a diastereoisomer, or a tautomer thereof, or a mixture thereof in any proportion according to claim 1.

5. The medicament according to claim 4, wherein the medicament is topical, oral or injectable.

6. The medicament according to claim 4, wherein the drug further comprises a pharmaceutically acceptable carrier or excipient.

7. The medicament according to claim 4, wherein the medicament is in a solid dosage form, a liquid dosage form or a semi-solid dosage form.

8. A compound represented by formula A, or a pharmaceutically acceptable salt, a solvate, an enantiomer, a diastereoisomer, or a tautomer thereof, or a mixture thereof in any proportion: 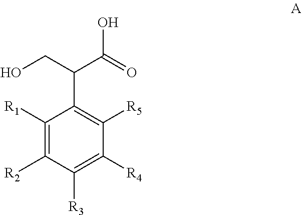
wherein, R₁-R₅ are each independently selected from —H or —OH;
provided that at least two of R₁-R₅ are selected from —OH.

9. The compound or a pharmaceutically acceptable salt, a solvate, an enantiomer, a diastereoisomer, or a tautomer thereof, or a mixture thereof in any proportion according to claim 8, wherein two or three of R₁-R₅ are selected from —OH.

10. The compound or a pharmaceutically acceptable salt, a solvate, an enantiomer, a diastereoisomer, or a tautomer thereof, or a mixture thereof in any proportion according to claim 8, wherein the compound is selected from the following compounds represented by formulas III-IV:

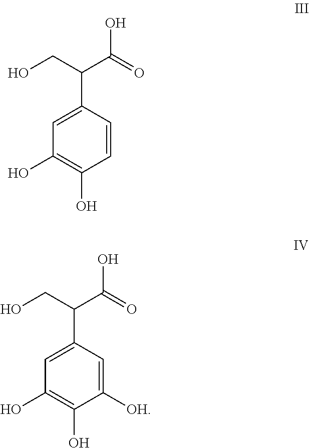 [SMILES: O=C(O)C(CO)c1ccc(O)c(O)c1 ; O=C(O)C(CO)c1cc(O)c(O)c(O)c1]

11. The medicament according to claim 4, wherein the medicament is in a dosage form of powder, tablet, coated tablet, granule, capsule, solution, emulsion, suspension, injection, spray, aerosol, powder for inhalation, lotion, liniment, ointment, plaster, paste, gel, or patch.
